(19) United States
(12) Patent Application Publication
(10) Pub. No.: US 2019/0307758 A1
(43) Pub. Date: Oct. 10, 2019

(54) TRPA1 ANTAGONISTS FOR USE IN THE TREATMENT OF ATOPIC DERMATITIS

(71) Applicant: GALDERMA RESEARCH & DEVELOPMENT, Biot (FR)

(72) Inventors: Gilles OUVRY, Biot (FR); Feriel HACINI-RACHINEL, Biot (FR); Christelle NONNE, Valbonne (FR)

(21) Appl. No.: 16/440,786

(22) Filed: Jun. 13, 2019

Related U.S. Application Data
(63) Continuation of application No. PCT/EP2017/082988, filed on Dec. 15, 2017.

(30) Foreign Application Priority Data
Dec. 16, 2016 (EP) 16306712.7
Dec. 16, 2016 (EP) 16306717.6

(51) Int. Cl. A61K 31/522; A61P 17/00; A61K 31/416; A61K 31/44; A61K 31/445; A61K 31/133; A61K 31/506

(52) U.S. Cl. CPC A61K 31/522; A61P 17/00; A61K 31/416; A61K 31/506; A61K 31/445; A61K 31/133; A61K 31/44

(57) ABSTRACT

The present invention relates to a TRPA1 receptor antagonist for use in preventing and/or treating the inflammatory component of atopic dermatitis.

### CROSS-REFERENCE TO RELATED PATENT APPLICATIONS

This application is a continuation of International Patent Application No. PCT/EP2017/082988, filed Dec. 15, 2017, which claims the benefit of and priority to EP Application No. 16306717.6, filed Dec. 16, 2016, and EP Application No. 16306712.7, filed Dec. 16, 2016. The entire disclosure of each application is incorporated by reference herein.

The present invention relates to the use of TRPA1 receptor antagonists for the prevention and/or treatment of the inflammatory component of atopic dermatitis. The present invention also relates to the use of a TRPA1 receptor antagonist to enhance the barrier function of the skin in a patient suffering from atopic dermatitis.

Atopic dermatitis and psoriasis belong to the most frequent inflammatory skin diseases.

Dermatitis derives from Greek language with “derma” meaning skin and “itis” meaning inflammation. Thus, dermatitis corresponds to skin inflammation that is classified in several specific and distinct types of dermatitis according to the localization, causes and symptoms thereof. Non exhaustive examples of dermatitis are atopic dermatitis, contact dermatitis, herpetiformis dermatitis, acrodermatitis, exfoliative dermatitis, perioral dermatitis, seborrheic dermatitis, eczema and hand eczema.

Atopic dermatitis is a condition of the epidermis which affects a large number of individuals genetically predisposed to atopy, including infants, children and pregnant women.

Atopic dermatitis (AD) is an increasingly common pruritic inflammatory skin disorder due to complex interactions between the genetic predispositions and environmental factors. Atopic dermatitis has a complex etiology that involves abnormal immunological and inflammatory pathways that include defective skin barrier, exposure to environmental agents and neuropsychological factors. The diagnosis of atopic dermatitis is based on clinical presentation of skin erythematous plaques, eruption and/or lichenification, typically in flexural areas accompanied by intense pruritus and cutaneous hypersensitivity. Histological examination reveals spongiosis, hyperkeratosis and parakeratosis in lesions, and marked epidermal hyperplasia, acanthosis and perivascular accumulation of lymphocytes and mast cells (mastocytes) in chronic lesions.

Although the exact pathophysiology of atopic dermatitis has not yet been clearly understood, it has been reported that atopic dermatitis has at least two major components corresponding to a damaged skin barrier and a deregulated immune response.

Existing AD treatments include anti-itch (antipruritic) therapies and anti-inflammatory therapies. They include multiple treatments: emollients to fight dry skin, disinfectants to fight bacterial infections, and topical corticosteroids or calcineurin inhibitors for control of inflammation. Despite these therapies, the itching may persist, which requires symptomatic treatment. Topical active agents such as urea, camphor or menthol are thus used. However, their efficacy remains limited (Darsow et al, Itch: Mechanisms and Treatment. Boca Raton (Fla.): CRC Press/Taylor & Francis, 2014. Chapter 3, Atopic Dermatitis).

As for anti-inflammatory therapies, they are effective in patients with intense flares and dense inflammatory cell infiltrates. Glucocorticoids, cyclosporin A, tacrolimus and pimecrolimus are the most effective treatments to date. However, such active agents have side effects and are partially efficient in severe AD.

There is thus a need for a treatment of atopic dermatitis which is effective, well tolerated and has the least possible side effects.

TRPA1 is a receptor of the superfamily of the transient receptor potential (TRP) channels. In mammals, TRPA1 is expressed in a subset of C-fibers which have cell bodies in nodose, dorsal root, and trigeminal ganglia and project to a variety of peripheral targets, including the skin. TRPA1 is a neuronal channel that has emerged as a key regulator of neuropeptide release and neurogenic inflammation. TRPA1 seems to be not expressed in keratinocytes (Zappia et al, PLOS ONE, Mar. 15, 2016). TRPA1 is robustly activated by a wide variety of exogenous irritants that cause pain and inflammation. The role of TRPA1 as a mediator of inflammatory pain and hypersensitivity is described for many years (Radresa et al. The Open Pain Journal 2013).

Recently, TRPA1 has been shown to be required for itch-evoked, Mrg-dependent signaling in sensory neurons and for itch evoked scratching induced by mast cell enkephalin peptide pruritogens and chloroquine (Bautista Annu Rev Physiol. 2013). Thus, TRPA1 may define a new signaling pathway that mediates histamine-independent itch (Bautista et al, Nat. Neurosci. 2014). TRPA1 channels are also required for Th2 cytokine-induced itch (IL31, IL13, TSLP for example), the same cytokines implicated in AD immune response (Wilson et al Cell 2013; Oh et al. J Immunol 2013 vol. 191 no. 11; Savinko et al. J Allergy Clin Immunol 2014). TRPA1 is also involved in chronic itch as described in mouse models of dry skin or AD (Wilson, J Neurosc 2013; Liu et al. FASEB 2013; Morita et al. Cell 2015). However these documents are only focused on the involvement of TRPA1 on the itch component.

Application WO2016/067143 describes the use of TRPA1 antagonists as analgesics and antipruritic agents.

Surprisingly, the Applicant has now discovered that, in a murine model of atopic dermatitis not amplified by scratching, TRPA1 antagonists decrease clinical signs (such as clinical scores and TransEpidermal Water Loss or TEWL), histological parameters (such as inflammatory cells in the dermis and epidermis thickness) and the production of Th2 proinflammatory cytokines. To assess the contribution of TRPA1 to AD inflammatory response, the inventors used a previously described model of AD based on repeated epicutaneous sensitizations (Spergel et al. JCI 1998; Staumont-Sallé et al. JEM 2015), with Dermatophagoïdes pteronyssinus (DERP) as antigen. In this model, AD skin lesions exhibit Th2-dominant inflammation characterized by dermal infiltration of CD4+ T cells and eosinophils with the deposition of eosinophil products and increased skin expression of Th2 cytokines.

In contrast, in a murine model of psoriasis, a TRPA1 antagonist does not reduce the inflammatory response involved in this pathology.

Thus, it is presently shown that TRPA1 is able to regulate the inflammatory component of atopic dermatitis, and this mechanism is independent from itching.

The Applicant has thus identified that TRPA1 antagonists can be used for effective prevention and/or treatment of the inflammatory component of atopic dermatitis, preferably via the topical route or preferably via the oral route. These compounds can also be used to enhance the skin barrier function in patients with atopic dermatitis.

Thus, the present invention relates to a TRPA1 receptor antagonist for use for preventing and/or treating the inflammatory component of atopic dermatitis.

The present invention also relates to a TRPA1 receptor antagonist for use for reinforcing the skin barrier function in a patient suffering from atopic dermatitis.

By “inflammatory component of atopic dermatitis”, it is meant an inflammation involving CD4+ lymphocytes, eosinophils, mast cells and Th2 cytokines. The inflammatory component of atopic dermatitis does not involve, and is different from, neurogenic inflammation (or neurogenic flare response). Indeed neurogenic inflammation occurs when stimulated sensory neurons release proinflammatory neuropeptides (such as substance P, calcitonin gene-related peptide (CGRP), neurokinin-A etc. . . . ) leading to localized vasodilatation and plasma extravasation (Grant et al. EJP 2005).

By “Th2 cytokines”, it is meant cytokines, notably produced by Th2 lymphocytes and keratinocytes, chosen from TSLP (Thymic Stromal Lymphopoietin), IL4, IL5, IL6 and IL13. Preferably, the Th2 cytokines are TSLP and IL4.

Said inflammatory component can thus be evaluated by measuring the amounts of CD4+ lymphocytes, eosinophils and mast cells which are present, and/or by measuring the amounts of Th2 cytokines in skin. These measurements can be carried out by any method known to those skilled in the art. For example, the cells may be quantified by histology. Cytokines may be evaluated by qRT-PCR.

The term “barrier function” or “skin barrier” is understood to mean the protective role of epidermal cells, especially the stratum corneum, with respect to the environment (i.e. water loss, physical and/or chemical aggressions and infectious agents).

The barrier function can be evaluated by means of the Transepidermal Water Loss test (or TEWL) and/or by the histological evaluation of epidermis thickness. By Transepidermal Water Loss, it is meant the percentage of water that passes through the keratin materials (more precisely the stratum corneum) and evaporates at the skin surface. The protocol of measurement of the TEWL is detailed in the examples.

By “TRPA1 receptor antagonist” according to the present invention, it is meant any compound which selectively blocks or inactivates the TRPA1 receptor or the cascade of biological signals related to TRPA1. The TRPA1 receptor antagonist of the invention binds to the TRPA1 receptor with high affinity. In any case, the affinity of the binding of said antagonist to TRPA1 is better than the one of the binding between said antagonist and another subtype of TRP superfamily receptors. Preferably, the affinity of the binding of said antagonist to TRPA1 is at least 100 fold higher than the one of the binding between said antagonist and another subtype of TRP superfamily receptors. Preferably, the other TRP superfamily receptor subtypes are the TRPV subtypes, preferably TRPV1, TRPV3 and/or TRPV4.

The human TRPA1 receptor has the sequence O75762 in Uniprot. It is a chemosensor expressed on the surface of the plasma membrane. It consists of six transmembrane domains, and has 14 ankyrin repeats at the intracellular N-terminus. It is stated above that the TRPA1 receptor antagonist according to the present invention binds to the TRPA1 receptor with high affinity. By “high affinity”, it is meant an affinity for the human TRPA1 receptor between 1 and 500 nM.

Preferably, the TRPA1 receptor antagonist is a chemical molecule, a peptide, a protein, an aptamer or an antibody.

The tests for determining whether a compound is an antagonist of the TRPA1 receptor are described in particular in Radresa et al. The Open Pain Journal 2013. Preferably, the test for measuring TRPA1 functionality is via electrophysiology or Ca²⁺ fluorescence assays, as explained on page 147 (“Electrophysiology profiling assays”) of Radresa et al, 2013.

According to one embodiment, the TRPA1 receptor antagonist according to the invention is a peptide, a protein or an antibody. By “peptide”, it is meant an amino acid sequence comprising from 2 to 30 amino acids. By “protein”, it is meant an amino acid sequence comprising at least 31 amino acids, preferably 50 to 500 amino acids. By “antibody”, it is meant a substance composed of four polypeptide chains, namely two light chains and two heavy chains.

In another embodiment, the TRPA1 receptor antagonist according to the invention is an aptamer. Aptamers are a class of molecules that represent an alternative to antibodies in terms of molecular recognition. Aptamers are oligonucleotide sequences having the ability to virtually recognize any class of target molecules with high affinity and specificity. These ligands can be isolated by the Systematic Evolution of Ligands by Exponential Enrichment (SELEX) technology from a library of random sequences, as described in Tuerk C. and Gold L., 1990. The library of random sequences can be obtained by combinatorial chemical synthesis of DNA. In this library, each member is a linear, optionally chemically modified, oligomer of a single sequence. The possible modifications, uses and advantages of this class of molecules have been studied in Jayasena S. Dak., 1999. The peptide aptamers consist of a variable region of conformationally constrained antibody exposed by a platform protein such as thioredoxin A from E. coli (Colas et al., 1996). Then, after identifying the aptamers directed against the TRPA1 receptor as described above, those skilled in the art can readily select the ones inhibiting the TRPA1 receptor.

Preferably, the TRPA1 receptor antagonist is a chemical molecule. More preferably, the antagonist of the TRPA1 receptor according to the invention is selected from:
the compounds of formula (I), their salts, their tautomers and their enantiomers:

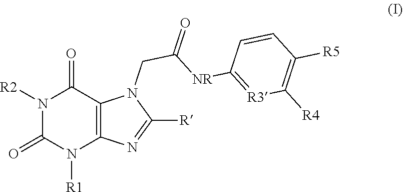

wherein R, R′, R1, R2 and R5 are identical or different, and are chosen from a hydrogen atom and a C1 to C12 alkyl radical;
R3 is a carbon atom or a heteroatom, preferably a nitrogen atom; and
R4 is a hydrogen atom or a radical (A):

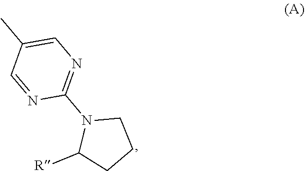

wherein R″ is a hydrogen atom or a C1-C12 alkyl radical; The compounds of formula (I) are described in WO2012/050641.
the compounds of formula (II), their salts, their tautomers and their enantiomers:

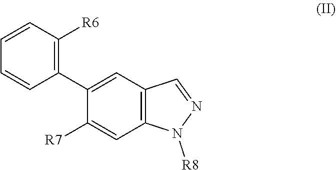

wherein R6, R7 and R8 are identical or different, and are chosen from a hydrogen atom, a C1 to C12 alkyl radical, a —CF3 radical and a —O—R9 radical, wherein R9 is a C1 to C12 alkyl radical;
The compounds of formula (II) are described in Rooney et al, 2014 (J Med Chem. 2014 Jun. 26; 57(12):5129-40. Discovery, Optimization, and Biological Evaluation of 5-(2-(Trifluoromethyl)phenyl)indazoles as a Novel Class of Transient Receptor Potential A1 (TRPA1) Antagonists).
the compounds of formula (III), their salts, their tautomers and their enantiomers:

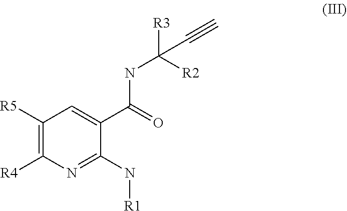

wherein
R1 is (C1-C6) alkyl, cyclo(C3-C6) alkyl, halo(C1-C6) alkyl, or phenyl, wherein said cyclo(C3-C6) alkyl, or phenyl is unsubstituted or substituted with 1 or 2 substituent(s) each independently being halogen, (C1-C2) alkyl or (C1-C2)alkoxy;
R2 is (C1-C2) alkyl;
R3 is (C1-C2) alkyl;
R4 is H, halogen, (C1-C2)alkoxy or halo(C1-C2)alkyl; and
R5 is H, halogen, (C1-C2)alkyl, (C1-C2)alkoxy, halo(C1-C2)alkyl, halo (C1-C2)alkoxy or (C1-C2)alkylthio.

The compounds of formula (III) are described in WO2014/053694.

Preferably the compound of formula (III) comprises H as R4, halogen as R5, and a substituted cyclo(C3-C6) alkyl as R1. Preferably it is the compound of formula (III′):

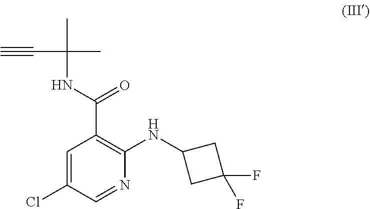 [SMILES: C#CC(C)(C)NC(=O)c1cc(Cl)cnc1NC1CC(F)(F)C1]

the compounds of formula (IV), their salts, their tautomers and their enantiomers:

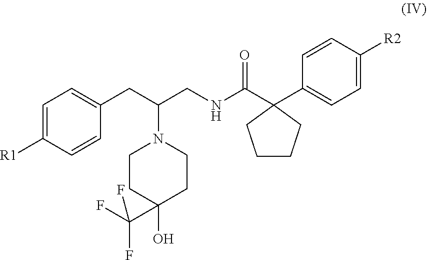

wherein R1 and R2 are each independently selected from H, halogen, OH, CH3, CF3 OCH3 and CN.

The compounds of formula (IV) are described in WO2016/067143.

Preferably the compound of formula (IV) comprises H as R1 and halogen as R2.
the compounds of formula (V), their salts, their tautomers and their enantiomers:

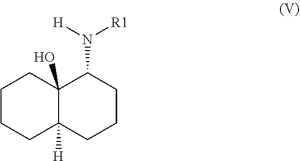

wherein R1 is selected from the group consisting of (CHR2)nC5-C10 heterocyclyl, (CHR2)nC6-C10 aryl, (CHR2)nC3-C10 cycloalkyl, or (C1-C6) alkyl; said alkyl, cycloalkyl, heterocyclyl and aryl being optionally substituted with 1 to 3 groups of R3; and n is an integer from 0 to 20; and
R2 and R3, when present, are each independently methyl or halogen.

The compounds of formula (V) are described in WO2011/043954.

Preferably the compound of formula (V) comprises (CHR2)nC6-C10 aryl, with n being 0, as R1, said aryl being substituted by one group of R3, R3 being a halogen. Preferably it is the compound of formula (V′):

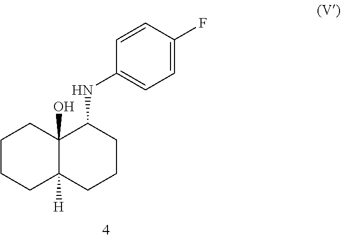 [SMILES: O[C@]12CCCC[C@@H]1CCC[C@H]2Nc1ccc(F)cc1]

the compounds of formula (VI), their salts, their tautomers and their enantiomers:

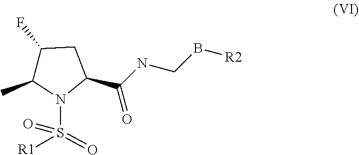

wherein
B is a 6-membered heteroaryl, wherein the 6-membered heteroaryl is unsubstituted or substituted with one or more groups independently selected from halogen, CN, (C1-C6) alkyl, (C1-C6) haloalkyl, O(C1-C6)alkyl, O(C1-C6)haloalkyl, 5 or 6-membered heteroaryl, (C3-C7)cycloalkyl, and 4, 5, 6 or 7-membered heterocyclyl; and wherein any of the 5 or 6-membered heteroaryl, (C3-C7)cycloalkyl, or 4, 5, 6 or 7-membered heterocyclyl groups is unsubstituted or substituted with one or more groups independently selected from halogen, CN, (C1-C6) alkyl, (C1-C6)haloalkyl, O(C1-C6)alkyl and O(C1-C6)haloalkyl;
R1 is phenyl or heteroaryl, wherein each phenyl or heteroaryl is unsubstituted or substituted with one or more groups independently selected from halogen, CN, (C1-C6)alkyl and (C1-C6)haloalkyl;
R2 is phenyl, (C3-C7)cycloalkyl, 5 or 6-membered heteroaryl, or 4, 5, 6 or 7-membered heterocycle, wherein any phenyl, (C3-C7)cycloalkyl, 5 or 6-membered heteroaryl, or 4, 5, 6 or 7-membered heterocycle is optionally substituted with one or more groups independently selected from halogen, CN, SF5, (C1-C6)alkyl, (C1-C6)haloalkyl, O(C1-C6)alkyl or O(C1-C6)haloalkyl;

The compounds of formula (VI) are described in WO2016/128529.

Preferably the compound of formula (VI) comprises a substituted phenyl as R1; as B, a 6-membered heteroaryl substituted by a (C1-C6) haloalkyl; and as R2, a 6-membered heteroaryl substituted by a (C1-C6)haloalkyl. Preferably it is the compound of formula (VI′):

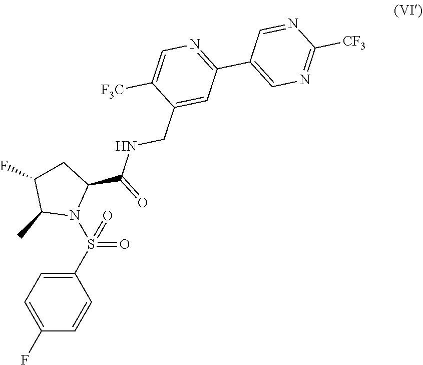 [SMILES: C[C@H]1[C@H](F)C[C@@H](C(=O)NCc2cc(-c3cnc(C(F)(F)F)nc3)ncc2C(F)(F)F)N1S(=O)(=O)c1ccc(F)cc1]

the compounds of formula (VII), their salts, their tautomers and their enantiomers:

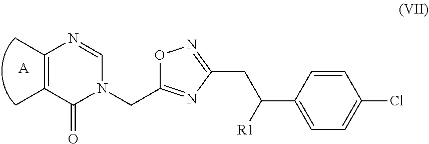

wherein
R1 is selected from H, OH, OMe or halogen; and
A is selected from the following:

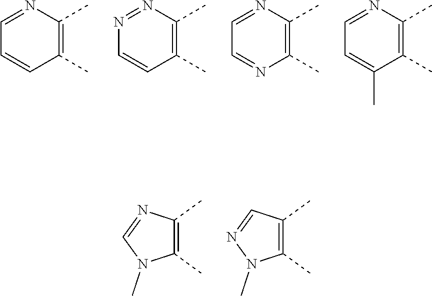

The compounds of formula (VII) are described in J. Med Chem 2016, 59, 2794.

Preferably the compound of formula (VII) comprises H as R1, and as A:

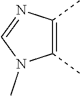

Preferably it is the compound of formula (VII′):

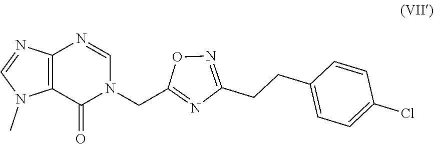 [SMILES: Cn1cnc2ncn(Cc3nc(CCc4ccc(Cl)cc4)no3)c(=O)c21]

the compounds of formula (VIII), their salts, their tautomers and their enantiomers:

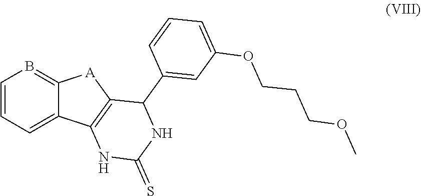

wherein A is:
a heteroatom, preferably oxygen, or
—C(═O)—,

### and B is:

a carbon atom, optionally linked to a halogen, preferably fluoride, or
a heteroatom, preferably nitrogen.

Such compounds are described in Copeland et al (Bioorganic & Medicinal Chemistry Letters 24 (2014) 3464:3468, Development of novel azabenzofuran TRPA1 antagonists as in vivo tools).

Preferably, the compound of formula (VIII) is of the following conformation (VIII′):

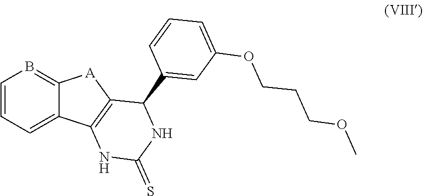

Preferably, the compound of formula (VIII) comprises, as A, an oxygen atom, and as B, a nitrogen atom or a carbon atom (not linked to a halogen).

Preferably, the compound of formula (VIII) comprises, as A, an oxygen atom, and as B, a nitrogen atom.
and the compound of the following formula (IX), their salts, their tautomers and their enantiomers:

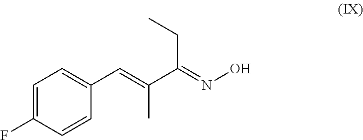

Compound (IX) is described in Pryde et al (J Name, 2013, The discovery of a potent series of carboxamide TRPA1 antagonists).

The term “C1 to C12 alkyl radical” means a substituted or unsubstituted, linear or branched, optionally cyclic, alkyl radical comprising from 1 to 12 carbon atoms. The C1 to C12 alkyl radical may be substituted with a group or an atom selected from halogen, —OH and —CF3. Preferably, the C1 to C12 alkyl radical is unsubstituted. Preferably, the C1 to C12 alkyl radical is a C1 to C6 alkyl radical (also called “(C1-C6) alkyl”). Preferably, it is chosen from methyl, ethyl, n-propyl, i-propyl, cyclopropylmethyl, n-butyl, 2-butyl, t-butyl, n-pentyl, i-pentyl and n-hexyl radicals. By “(C1-C2) alkyl”, it is meant methyl or ethyl.

By “cyclo(C3-C6)alkyl”, “cyclo(C3-C7)alkyl” (or (C3-C7)cycloalkyl) and “cyclo(C3-C10)alkyl” (or (C3-C10)cycloalkyl), it is meant a substituted or unsubstituted cyclic alkyl radical comprising from 3 to 6 carbon atoms, or 3 to 7 carbon atoms, or 3 to 10 carbon atoms, respectively. Preferably, the cyclo(C3-C6)alkyl or cyclo(C3-C7)alkyl or cyclo(C3-C10)alkyl is unsubstituted. Preferably, it is chosen from cyclopropyl, cyclopentyl and cyclohexyl.

By “(C1-C2) alkoxy”, it is meant methoxy or ethoxy.

By “halo(C1-C2) alkoxy”, it is meant methoxy or ethoxy in which at least one hydrogen is substituted by a halogen.

By (C1-C2)alkylthio, it is meant methylthio or ethylthio.

By “halo(C1-C2) alkyl”, it is meant methyl or ethyl in which at least one hydrogen is substituted by a halogen.

By “halo(C1-C6) alkyl” or “(C1-C6)haloalkyl”, it is meant a (C1-C6) alkyl in which at least one hydrogen is substituted by a halogen.

By “C5-C10 heterocyclyl”, it is meant a saturated or aromatic carbon cycle, comprising from 5 to 10 carbon atoms, and comprising at least a heteroatom.

By “C6-C10 aryl”, it is meant an aromatic carbon cycle, comprising from 6 to 10 carbon atoms. Preferably it is a phenyl.

By “heteroaryl”, it is meant an aromatic heterocycle. Preferably, the heteroatom is nitrogen. Preferably, the heteroaryl is pyridine or pyrimidine.

The term “heteroatom” means an atom such as oxygen, nitrogen, sulfur or phosphorus. Preferably, the heteroatom according to the invention is nitrogen.

The term “halogen” means preferably a bromide, chloride, fluoride or iodine atom. Preferably, the halogen is a chloride or a fluoride.

The term “salt” is understood to mean a salt of a compound of formula (I) or (II) with an alkaline earth metal, preferably a sodium salt, a potassium salt or a magnesium salt.

Preferably, the antagonist of the TRPA1 receptor of formula (I) is as follows:

R and R′ are each hydrogen;
R1 and R2 are each a C1 to C12 alkyl radical, preferably methyl;
R3 is a carbon atom or a heteroatom, preferably a nitrogen atom;
R4 is a hydrogen atom or a radical (A):

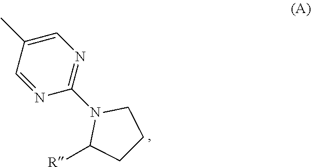

wherein R″ is a C1 to C12 alkyl radical, preferably methyl; and
R5 is a hydrogen atom or a C1-C12 alkyl radical, preferably a 2-butyl radical.

The TRPA1 receptor antagonist of formula (I) as follows:

R, R′ and R5 are each hydrogen;
R1 and R2 are each methyl;
R3 is a nitrogen atom; and
R4 is a radical (A):

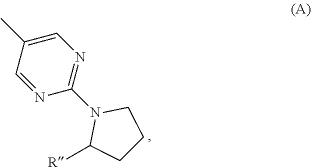

wherein R″ is methyl,
is called “compound A” in the present application.

The TRPA1 receptor antagonist of formula (I) as follows:

R and R′ are each hydrogen;
R1 and R2 are each methyl;
R3 is a carbon atom;
R4 is hydrogen; and
R5 is a 2-butyl radical, is called “compound D” in the present application.

Preferably, the antagonist of the TRPA1 receptor of formula (II) is as follows:

### R6 is —CF3;

R7 is a C1 to C12 alkyl radical, preferably methyl, or R7 is a radical —O—R9, where R9 is a C1 to C12 alkyl radical, preferably cyclopropylmethyl; and
R8 is hydrogen.

The TRPA1 receptor antagonist of formula (II), as follows:

### R6 is —CF3;

R7 is methyl; and
R8 is hydrogen, is referred to as “compound B” in the present application.

The TRPA1 receptor antagonist of formula (II), as follows:

### R6 is —CF3;

R7 is —O—R9, wherein R9 is cyclopropylmethyl; and
R8 is hydrogen, is referred to as “compound C” in the present application.

More particularly, the TRPA1 receptor antagonist according to the invention is administered in the form of a composition, preferably a dermatological composition. Preferably, the TRPA1 receptor antagonist is administered topically to the skin. Preferably, the composition, in particular a dermatological composition, comprises, in a physiologically acceptable medium, at least one TRPA1 receptor antagonist according to the invention. Preferably, said composition comprises from 0.001% to 10% by weight of the TRPA1 receptor antagonist according to the invention, relative to the total weight of composition.

Preferably, the subject treated with the TRPA1 receptor antagonist according to the invention is a mammal, preferably a human.

By “physiologically acceptable medium” is meant a medium compatible with topical administration.

By topical route, the composition, in particular the dermatological composition, according to the invention, can be present in all galenic forms normally used for topical administration. By way of non-limiting example of topical preparations, preparations may be mentioned in liquid, pasty or solid form and, more particularly, in the form of ointments, aqueous, aqueous-alcoholic or oily solutions, dispersions of the optionally two-phase lotion type, serum, aqueous, anhydrous or lipophilic gels, powders, soaked pads, syndets, wipes, sprays, foams, sticks, shampoos, compresses, washing bases, emulsions of liquid or semi-liquid consistency such as milk, obtained by dispersing a fatty phase in an aqueous phase (O/W) or inversely (W/O), a microemulsion, suspensions or emulsions of soft, semi-liquid or solid of white or colored cream type, gel or ointment, suspensions of microspheres or nanospheres or lipid or polymeric vesicles, or microcapsules, micro- or nanoparticles or of polymeric or gelled patches allowing a controlled release.

It is routine for those skilled in the art to adjust the nature and amount of the additional active ingredients and excipients in the pharmaceutical composition so as not to affect the desired properties thereof, notably with regard to the stability of the TRPA1 receptor antagonist according to the invention, and the route of administration considered.

The physiologically acceptable medium may comprise various excipients. By “excipient” it is meant an inert substance typically used as a diluent or carrier for the TRPA1 receptor antagonist according to the invention.

Emulsions such as oil-in-water (O/W) or water-in-oil (W/O) systems, as well as a base (vehicle or support) for the topical formulation, may be chosen so as to ensure efficacy of the active ingredients and/or to avoid allergic and irritant reactions. The compositions may comprise an emulsifier. Non-limiting examples of emulsifiers useful in this regard include glycol esters, fatty acids, fatty alcohols, fatty acid esters of glycols, fatty esters, fatty ethers, glycerine esters, propylene glycol esters, polyethylene glycol fatty acid esters, fatty acid esters of polypropylene glycol, sorbitol esters, esters of sorbitan anhydrides, copolymers of carboxylic acids, glucose esters and ethers, ethoxylated ethers, ethoxylated alcohols, alkyl phosphates, polyoxyethylene phosphate ethers, fatty acid amides, acyl lactylates, soaps and mixtures thereof. Specific non-limiting examples of emulsifiers useful in the present compositions include polyethylene glycol-20 sorbitan monolaurate (polysorbate-20), polyethylene glycol, soybean sterol, steareth-2, steareth-20, steareth-21, ceteareth-20, glucose methyl ether distearate PPG-2, ceteth-10, polysorbate-80, cetyl phosphate, potassium cetyl phosphate, diethanolamine cetyl phosphate, polysorbate-60, glyceryl stearate, PEG-100 stearate, tragacanth gum and mixtures thereof.

The lotions useful in the present compositions may be suspensions of powdered material in an aqueous or alcoholic base.

Ointments are oleaginous compositions that contain little or no water (anhydrous).

The compositions can also be in the form of gels. In this regard, the compositions may comprise a gelling agent and/or a thickener. Suitable gelling and/or thickening agents which may be useful in the present compositions include aqueous thickeners, such as neutral, anionic and cationic polymers, and mixtures thereof. Examples of polymers which may be useful in the present compositions include carboxyvinyl polymers, such as carboxypolymethylene. A preferred thickener is a carbomer, for example a Carbopol® polymer from Noveon Inc. Other examples of polymers useful in this regard include hydrophilic/hydrophobic graft copolymers, such as polymers formed as a mixture of polystyrene/microsponge/Carbopol®. Such a polymer in this respect is a dimethyl acrylamide/acrylic acid/polystyrene ethyl methacrylate copolymer, for example a copolymer of the Pharmadur® brand as available from Polytherapeutics.

Other non-limiting examples of suitable thickeners include cellulosic polymers such as arabic gum, tragacanth gum, locust bean gum, guar gum, hydroxypropylguar, xanthan gum, cellulose gum, sclerotium gum, carrageenan gum, karaya gum, cellulose, rosin, methylcellulose, hydroxyethylcellulose, hydroxypropylcellulose, hydroxymethylcellulose, hydroxypropylmethylcellulose, methyl hydroxyethylcellulose, cetylhydroxyethylcellulose, carboxymethylcellulose, corn starch, hydroxypropyl phosphate starch, PEG-150/alkoxy stearyl alcohol/SMDI copolymer, PEG-180/laureth-50/TMMG copolymer, acrylic acid/acrylamidomethylpropane sulfonic acid copolymer, acrylate/C10-30 acrylate copolymer, acrylate/beheneth-25 methacrylate copolymer, acrylate/steareth-20 methacrylate copolymer, acrylate/stearth-20 copolymer, acrylate/VA copolymer, ammonium acryloyldimethyltaurate/beheneth-25 methacrylate, ammonium acryloyldimethyltaurate/VP copolymer, caprylic/capric triglyceride (and) sodium acrylate copolymer, propylene glycol alginate, dimethicone and mixtures thereof. Other thickeners and/or gelling agents, such as polyacrylic polymers, may be used.

Alternatively, the TRPA1 receptor antagonist according to the invention is administered in the form of a composition for oral route. Preferably, the oral composition comprises, in an orally acceptable medium, at least one TRPA1 receptor antagonist according to the invention. Preferably, said composition comprises from 0.001% to 10% by weight of the TRPA1 receptor antagonist according to the invention, relative to the total weight of composition.

Preferably, the subject treated with the TRPA1 receptor antagonist according to the invention is a mammal, preferably a human.

By “orally acceptable medium” is meant a medium compatible with oral administration.

By oral route, the composition according to the invention, can be present in all galenic forms normally used for oral administration. Thus, the oral composition according to the invention may have a liquid, pasty or solid form and, more particularly, in the form of ointments, aqueous, aqueous-alcoholic or oily solutions or emulsions, syrups or semi-liquid consistency such as milk, obtained by means of the usual methods used for the manufacture of emulsions, tablets, capsules, sugar-coated tablets, capsules, gels or hydrogels enabling controlled release.

The orally acceptable medium may comprise various excipients. By “excipient” it is meant an inert substance typically used as a diluent or carrier for the TRPA1 receptor antagonist according to the invention.

Examples of excipients which may be used include binders, disintegrants, lubricants, coating agents, plasticizers, compression agents, wet granulating agents, as well as sweeteners, which are all known to those skilled in the art to which the invention relates. All the following examples are given for illustrative and non-limiting purposes. Binders are used where appropriate to help keep the ingredients together. Examples of binders include carbopol, povidone and xanthan gum. Lubricants are generally used in the manufacture of compositions by direct compression in order to prevent the compacted powder mass from sticking to the apparatus during the tabletting or encapsulation process. Examples of lubricants include calcium stearate, magnesium stearate, stearic acid, sodium stearyl fumarate, and vegetable fatty acids. Disintegrants help break the compacted mass when placed in a fluid environment. Examples of disintegrants include sodium croscarmellose, crospovidone, gellan gum, hydroxypropyl cellulose, starch and sodium starch glycolate. The coating agents are used to control the solubility of the drug. Examples of coating agents include carrageenan, cellulose phthalate acetate, ethylcellulose, gellan gum, maltodextrins, methacrylates, methylcellulose, microcrystalline cellulose and shellac. Plasticizers are used to control the release rate of the drug from the dosage form. Examples of plasticizers include citrate esters, dibutyl sebacate, diethyl phthalate, polyvinyl acetate phthalate and triacetin. Compression agents include calcium carbonate, dextrose, fructose, guar gum, honey, lactose, maltodextrin, maltose, mannitol, microcrystalline cellulose, sorbitol, starch and sucrose. Wet granulating agents include calcium carbonate, lactose, maltodextrin, mannitol, microcrystalline cellulose, povidone and starch. Sweeteners include aspartame, dextrose, fructose, honey, lactose, maltodextrin, maltose, mannitol, monoammonium glycyrrhizinate, sorbitol, sucralose, and sucrose. Of course, the nature and the quantity of the ingredients used are adapted to the galenic forms envisaged.

The invention will be better understood on reading the following illustrative and non-limiting examples.

In the following examples, reference is made to the following figures:

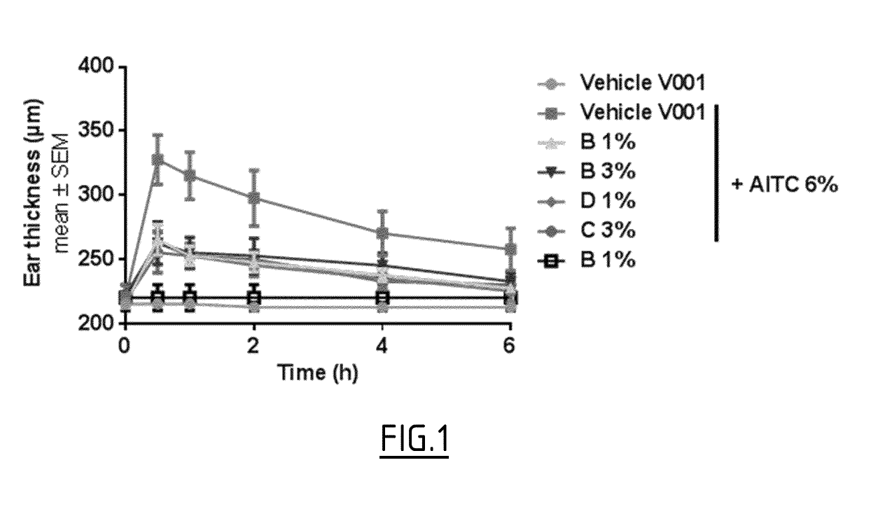

FIG. 1: Topical activity of TRPA1 antagonists of the invention

Test antagonists B to D were formulated at 1% or 3% in an acetone vehicle, applied to the mouse ear skin (10 μl/ear both faces) and left for 1 hour. Then, 10 μl of a 6% solution of allyl isothiocyanate (AITC) in acetone (vehicle 001) was applied to the same location, and ear thickness was measured at T0, 30 min, 1 h, 2 h, 4 h and 6 h.

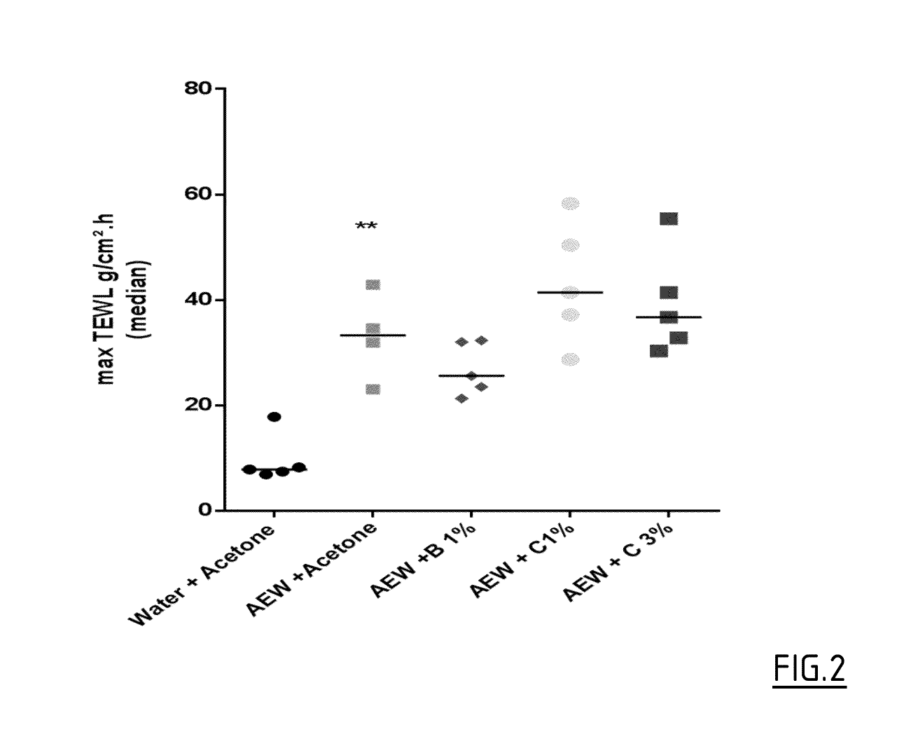

FIG. 2: TEWL in a dry-skin model following TRPA1 antagonist application

Mice were treated twice daily, on the shaved upper part of the back (the nape), with a cotton swab immersed in either a 1:1 mixture of acetone and ether (“AEW treatment”) or water (control) for 15 s, followed by water for 30 s for a duration of 8 days. Topical treatment with compound B at 1%, or compound C at 1% or 3%, 100 μl at the same site, was performed 1 hour before AEW treatment or control treatment. The site of treatment was, for the mice, hard to access for to scratching.

TEWL is measured each day, during 8 days, using a Tewameter on a vigil animal. The Tewameter® probe measures the density gradient of the water evaporation from the skin indirectly by the two pairs of sensors (temperature and relative humidity) inside a hollow cylinder. A microprocessor analyses the values and expresses the evaporation rate in g/h/m².

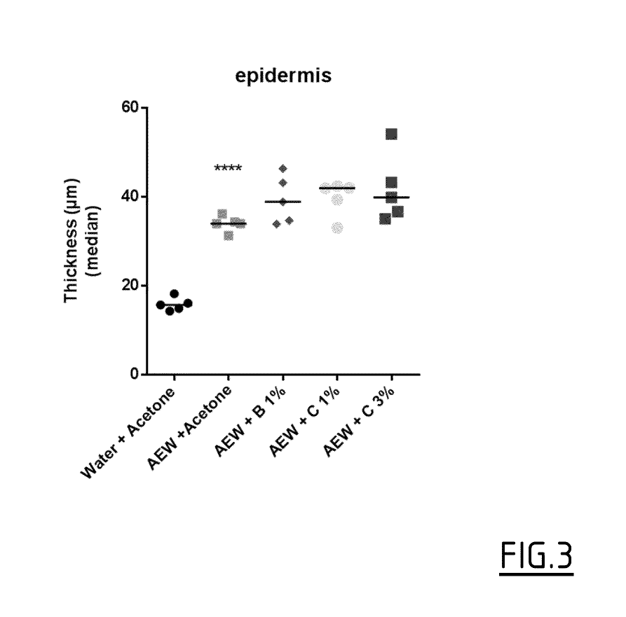

FIG. 3: Epidermis thickness in a dry-skin model following TRPA1 antagonist application

The treatment protocol is the same as for FIG. 2.

Epidermis thickness is measured the last day (Day 8) by histological morphometric analysis on HE-stained 6 μm-thick sections from previously formal saline-fixed skin samples.

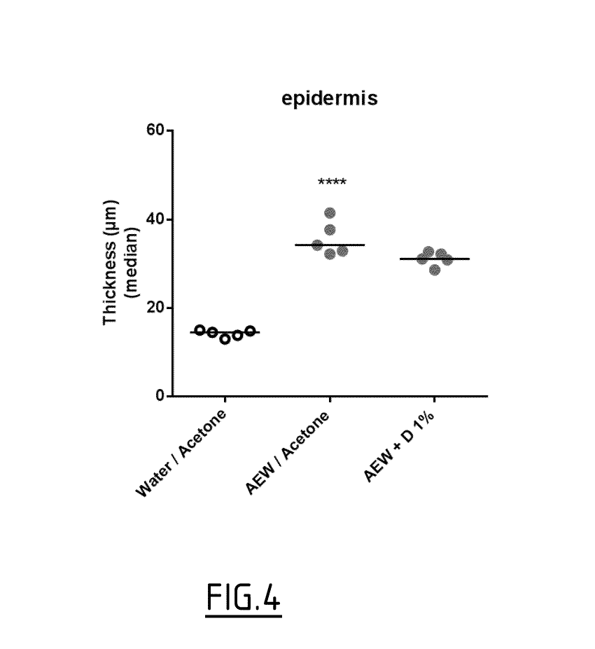

FIG. 4: Epidermis thickness in a dry-skin model following TRPA1 antagonist application

The treatment protocol is the same as for FIG. 2, except that compound D at 1% was topically applied, instead of compounds B or C, 1 h before AEW treatment or control treatment.

Epidermis thickness is measured according to the protocol of FIG. 3.

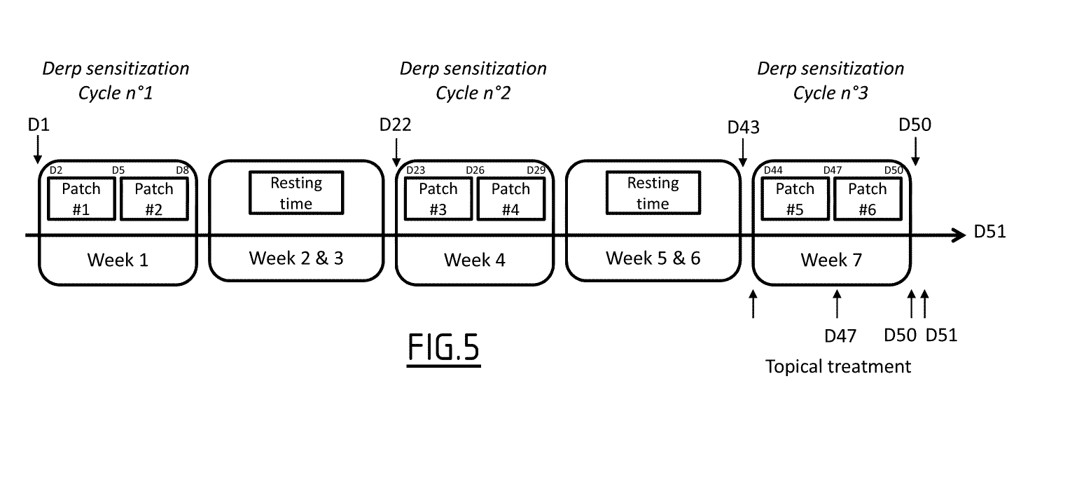

FIG. 5: Chronogram of mouse AD-patch model

Female Balb/c mice were treated with Dermatophagoïdes pteronyssinus (DERP) allergen in 3 cycles of epicutaneous sensitization (patch) on abdominal skin with 2 patches applied twice weekly followed by 2 weeks of rest, during 7 weeks. Antagonists B or D were formulated at 1% in an acetone vehicle, applied to the sensitized-skin 2 hours before DERP-patches installation. Topical treatments with antagonists or a corticosteroid (used as a control) were only performed 3 times in the 3^{rd} and last week of sensitization (at days 44, 47 and 50).

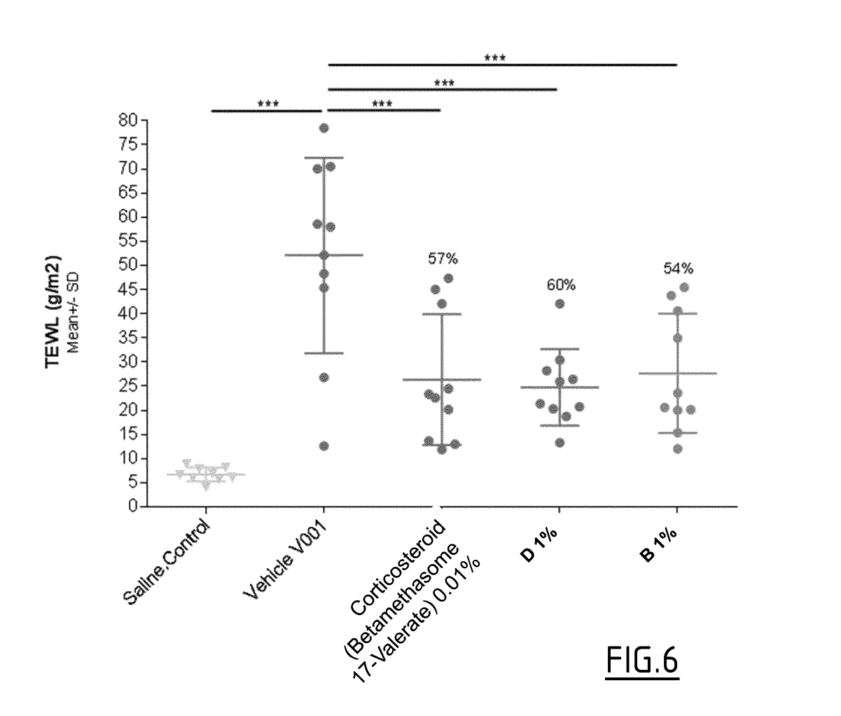

FIG. 6: TEWL in an AD-patch model following TRPA1 antagonist application

The treatment protocol is described in FIG. 5.

TEWL is measured the last day (Day 51) according to the protocol of FIG. 2.

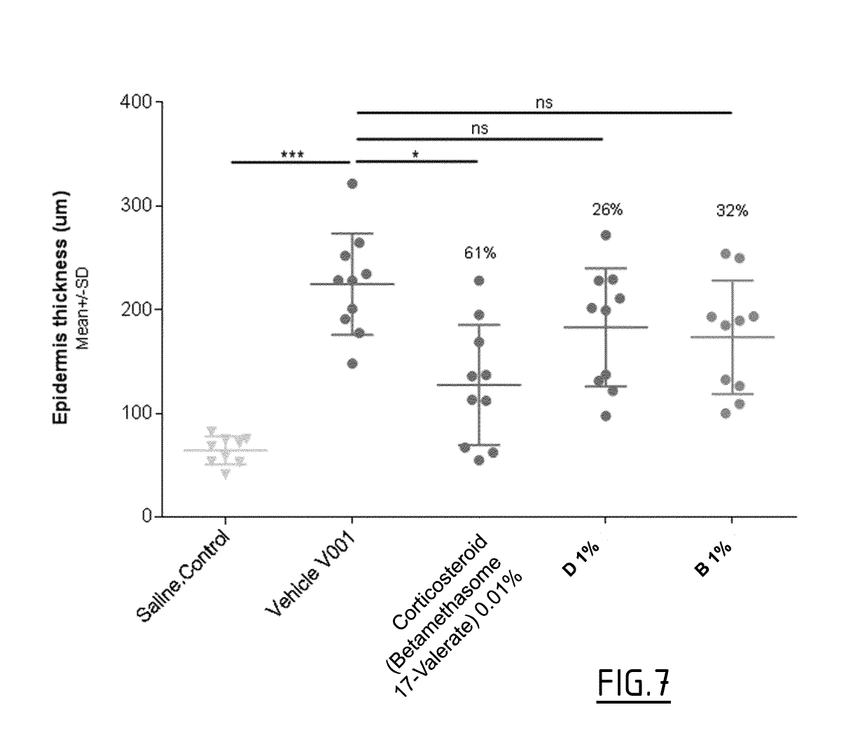

FIG. 7: Epidermis thickness in an AD-patch model following TRPA1 antagonist application

The treatment protocol is described in FIG. 5.

On day 51, animals are sacrificed and skin is removed and fixed in formalin followed by embedment in paraffin, sectioning and staining with hematoxylin and eosin. Epidermis thickness is measured by averaging the values of measurements of five independent fields of view.

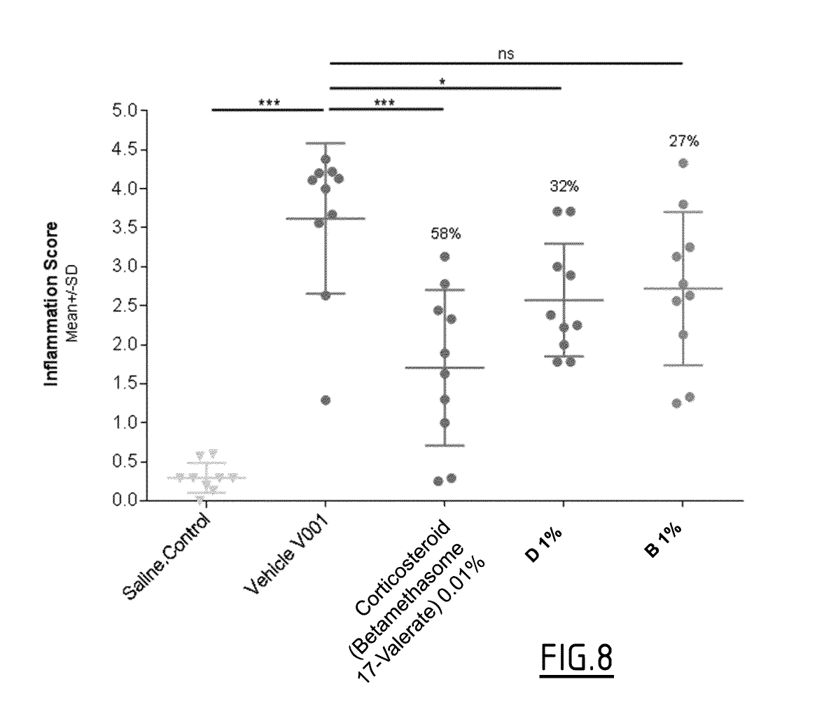

FIG. 8: Inflammation score in an AD-patch model following TRPA1 antagonist application

The treatment protocol is described in FIG. 5.

On day 51, animals are sacrificed and skin is removed and fixed in formalin followed by embedment in paraffin, sectioning and staining with hematoxylin and eosin. Inflammation score is quantified by averaging the values of 10 random fields of view per sample, scored for inflammation with a gradient (semi-quantitative scoring from 0 to 5).

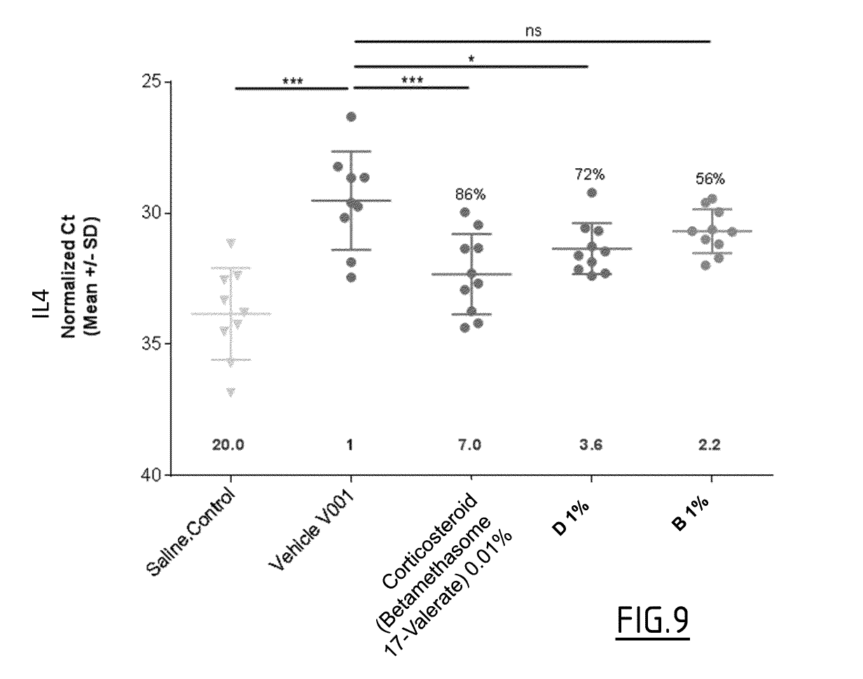

FIG. 9: IL4 mRNA expression in an AD-patch model following TRPA1 antagonist application

The treatment protocol is described in FIG. 5.

At day 51, gene expression of IL4 cytokine is quantified after RNA extraction from skin, RNA retro transcription and PCR using the TaqMan® Universal MasterMix of Applied Biosystem. cDNA of mice of group Vehicle 001 are used as comparator.

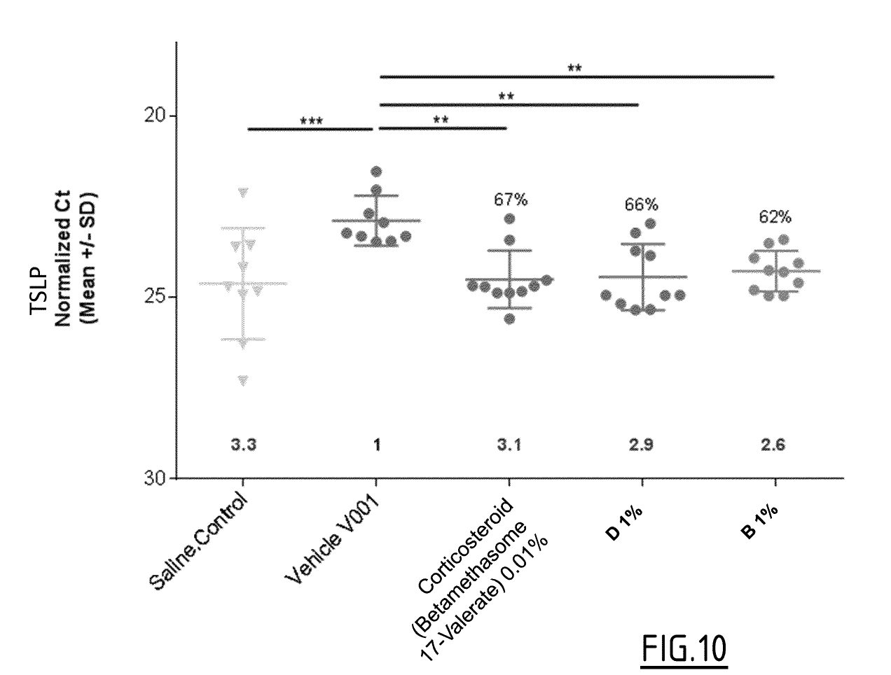

FIG. 10: TSLP mRNA expression in an AD-patch model following TRPA1 antagonist application

The treatment protocol is described in FIG. 5.

At day 51, gene expression of TSLP cytokine is quantified after RNA extraction from skin, RNA retro transcription and PCR using the TaqMan® Universal MasterMix of Applied Biosystem. cDNA of mice of group Vehicle 001 are used as comparator.

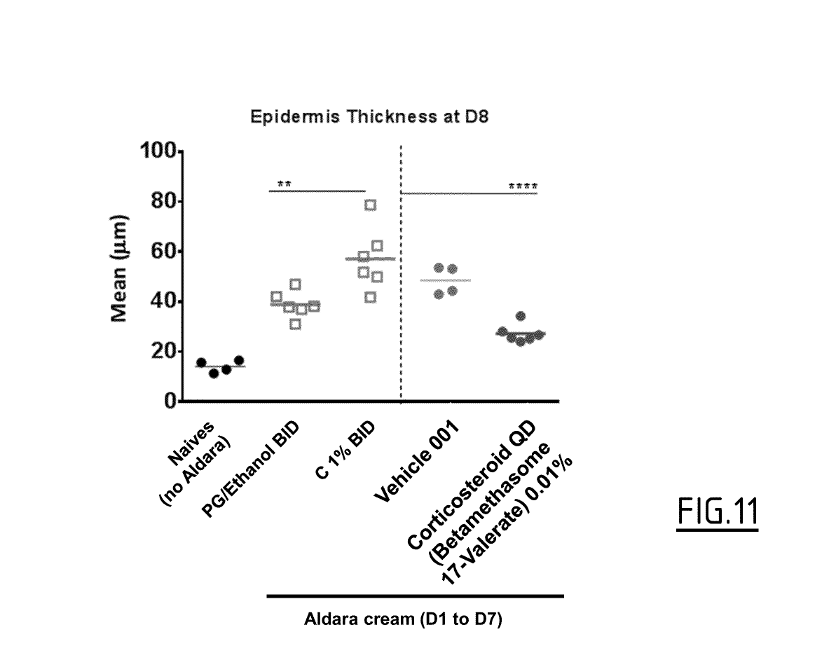

FIG. 11: Epidermis thickness in a psoriasiform model following TRPA1 antagonist application

The treatment protocol consists of a daily topical application of Aldara (3.18 mg of imiquimod) for 7 days on the shaved back skin of Balb/c mice. Compound C was dissolved in PG/EtOH at 1% and was applied twice daily 2 h before and 6 h after Aldara treatment. Corticosteroid was dissolved in acetone (Vehicle 001) at 0.01% and applied only 2 h before Aldara treatment.

Epidermis thickness is then measured at day 8 according to the protocol of FIG. 3.

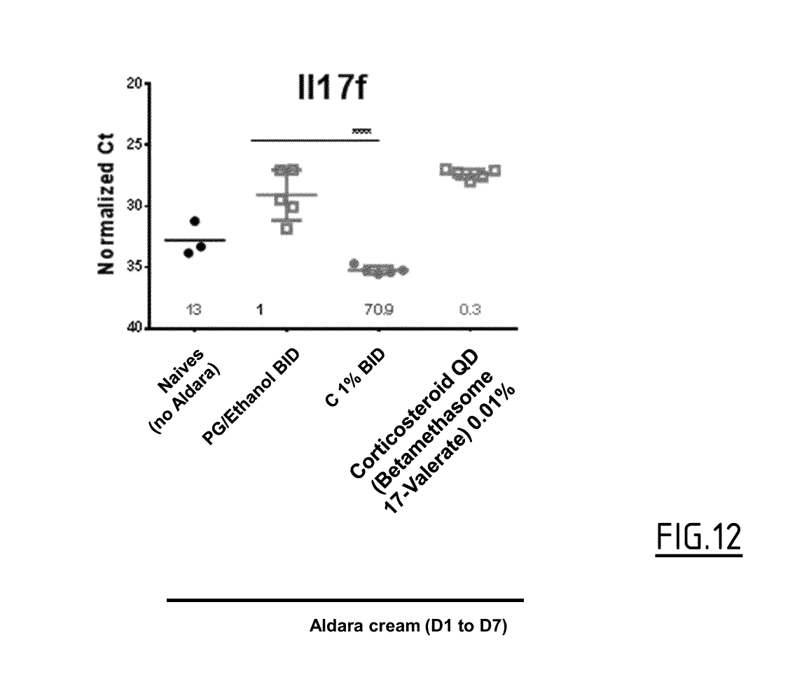

FIG. 12: IL17f mRNA expression in a psoriasiform model following TRPA1 antagonist application

The treatment protocol is described in FIG. 11.

At day 8, gene expression of IL17f cytokine is quantified after RNA extraction, RNA retro transcription and PCR using the TaqMan® Universal MasterMix of Applied Biosystem. cDNA of mice of group PG/EtOH are used as comparator

### EXAMPLE 1: STRUCTURES AND PROPERTIES OF DIFFERENT TRPA1 RECEPTOR ANTAGONISTS OF THE INVENTION

The compounds A to D of the invention have been described for their affinity for the different TRPA1 receptors (human, rat, murine or drosophila), as well as for TRPV and TRPM receptors subtypes, in different patent applications or publications (WO2013023102, Rooney et al. J Med Chem 2014, Wei et al Neuropharmacology 2010, Wei et al. Anesthesiology 2009).

Compound D is a known compound, useful as a pharmacological tool for studying TRPA1-dependent pain in animal models (Wei et al Neuropharmacology 2010, Wei et al. Anesthesiology 2009).

The results are as follows:

| | | | | Chembridge- |
|---|---|---|---|---|
| IC50 | A | B | C | 5861528 D |
| hTRPA1 | 93 nM | 15 nM | 26 nM | 14-18 μM |
| rTRPA1 | 101 nM | 89 nM | 190 nM | 230 nM |
| mTRPA1 | NA | 53 nM | 104 nM | NA |
| dTRPA1 | 102 nM | NA | NA | NA |
| hTRPV1 | >100 | 5.1 μM | 8.1 μM | NA |
| hTRPV3 | >300 | >30 | >30 | NA |
| hTRPV4 | 16 μM | NA | NA | NA |
| hTRPM8 | 19 μM | 9.2 μM | 4.4 μM | NA |
| | | Efficacy in | Low oral | Efficacy |
| | | pain models | bioavailability | described in pain |
| | | described in | described in rat | models in rat |
| | | rat and | Efficacy | |
| | | mouse (oral) | described in | |
| | | | pain model (ip) | |

### EXAMPLE 2: TOPICAL ACTIVITY OF TRPA1 RECEPTOR ANTAGONISTS OF THE INVENTION

A topical pharmacodynamics (PD) model was designed in mouse, in which test antagonists B to D were formulated at 1% or 3% in an acetone vehicle, applied to the ear skin and left for 1 hour. After this time, a 6% solution of allyl isothiocyanate (AITC), a TRPA1 agonist, in acetone (vehicle 001) was then applied to the same location and ear edema (via the ear thickness) was measured during 6 hours.

Results are shown in FIG. 1. They show that topically applied, compounds B, C and D at 1% or 3% decreased the TRPA1 agonist-induced ear edema.

This test validated the topical activity of the TRPA1 antagonists of the invention in mouse skin after a single application.

### EXAMPLE 3: STUDY OF DIFFERENT TRPA1 RECEPTOR ANTAGONISTS OF THE INVENTION IN AN AD-PATCH MODEL

AD inflammatory response was induced by epicutaneous sensitization with patches soaked with 100 μg of allergen in sterile saline (Dermatophagoïdes pteronyssinus or DERP) or with vehicle applied on abdominal skin 24 h after shaving and left on for three 1-wk periods (with patch renewal at midweek), with a 2-wk interval between applications. The presence of the patches and of a transparent occlusive dressing above, prevented licking, biting and scratching from mouse, therefore the inflammatory response in this model is not due to the scratch-itch cycle.

Antagonists B or D were formulated at 1% in an acetone vehicle, applied to the sensitized-skin 2 hours before DERP-patches installation. Topical treatments with antagonists or a corticosteroid (used as a control) were only performed 3 times in the 3^{rd} and last week of sensitization (at days 44, 47 and 50).

At the time of the last patch removal (day 51), skin samples and blood were collected.

Skin was collected for histological, immunohistological analyses and real-time PCR after RNA extraction. Ig (IgE, and specific IgG) concentrations in serum were measured by ELISA

FIG. 5 indicates the chronogram of treatments.

Results are shown in FIGS. 6 to 10. Antagonists D and B decrease all studied parameters. The TEWL-increase, a clinical sign of skin barrier dysfunction, is significantly decreased (−60% and −54% respectively). Another parameter of skin barrier defect, the epidermis acanthosis, is partially restored with TRPA1 antagonists (−26% and −32% respectively of epidermis thickness). The inflammatory parameters are also impacted such as inflammatory scores (−32% and −27% respectively) and expression of Th2 cytokines as demonstrated with IL4 and TSLP mRNA analysis (−56% to −66%). The efficacy of TRPA1 antagonists is fairly close to the corticosteroid treatment.

### EXAMPLE 4: STUDY OF A TRPA1 RECEPTOR ANTAGONIST OF THE INVENTION IN A PSORIASIS MODEL

Imiquimod (IMQ) repeated topical application on mouse skin has been shown to induce a skin inflammation resembling the phenotype of human psoriasis (PLoS One. 2011). Indeed, it induces inflamed scaly skin lesions resembling psoriasis plaques. These lesions showed increased epidermal proliferation, abnormal differentiation, epidermal accumulation of neutrophils in microabcesses, neoangiogenesis and infiltrates consisting of CD4(+) T cells, CD11c(+) dendritic cells, and plasmacytoid dendritic cells. IMQ induced epidermal expression of IL-23, IL-17A, and IL-17F, as well as an increase in splenic Th17 cells (J Immunol. 2009 May 1; 182(9):5836-45).

Mice received a daily topical dose of 63.5 mg (14 μl) of commercially available IMQ cream (5%) (Aldara; 3M Pharmaceuticals) on the shaved back skin for 7 consecutive days.

In this model, topical administration of the TRPA1 antagonist compound C did not significantly reduce the imiquimod-induced epidermis thickness increase and IL-17 production by γδ T cells at day 8 (see FIGS. 11 and 12).

The difference of efficacy between the psoriasis model of example 4 and the AD-patch model of example 3 is explained by the main cytokines involved in each case. Indeed the imiquimod induced skin inflammation in mediated by the IL-23-IL-17 axis, with no contribution of Th2-related cytokines (IL-4, IL-13, TSLP . . . ).

These results are supported by recent data with TRPA1-deficient mice in the same Imiquimod model (Th1/17 model; oral presentation and abstract, Kemeny et al. ESDR September 2016) and in an Th1-oriented colitis model (Bertin et al. Gut 2016).

### EXAMPLE 5: STUDY OF TRPA1 RECEPTOR ANTAGONISTS OF THE INVENTION IN A DRY-SKIN MODEL, ON A SITE INDEPENDENT OF SCRATCHING

The model used was set up based on the publication of Wilson J. Neurosc 2013. Mice were treated twice daily with a cotton swab immersed in either a 1:1 mixture of acetone and ether or water for 15 s, followed by water for 30 s for a duration of 8 days (named “AEW treatment”).

Topical treatment with TRPA1 antagonists (i.e. compounds B, C or D at 1%, or compound C at 3%) at the same site was performed 1 hour before AEW treatment. The site of treatment was, for the mice, inaccessible to scratching.

Epidermis thickness and TEWL were measured.

The results are shown in FIGS. 2 to 4.

In this context where animals cannot scratch, chronic treatments with TRPA1 antagonists were unable to decrease the epidermis thickness and TEWL, in contrast to the results obtained by Wilson et al (J. Neurosc 2013).

These results indicate that the effect of TRPA1 antagonists of the invention upon epidermis thickness in the AEW model is scratching-mediated, and is independent of a direct effect of TRPA1 upon keratinocytes.

Because mice cannot scratch in the AD patch model, these results suggest that the effect of TRPA1 antagonists on skin barrier (measured by TEWL & epidermis thickness in the AD patch model) is due to the anti-inflammatory effect.

1. A method for preventing and/or treating the inflammatory component of atopic dermatitis in a subject in need thereof, comprising administering to the subject a TRPA1 receptor antagonist.

2. The method according to claim 1, wherein the inflammatory component of atopic dermatitis is an inflammation involving CD4+ lymphocytes, eosinophils, mast cells and Th2 cytokines.

3. The method according to claim 2, wherein the Th2 cytokines are such as Thymic Stromal Lymphopoietin, IL4, IL5, IL6 and IL13.

4. The method according to claim 1, wherein the TRPA1 receptor antagonist is administered topically to the skin.

5. The method according to claim 1, wherein the TRPA1 receptor antagonist is administered via oral route.

6. The method according to claim 1, wherein the TRPA1 receptor antagonist is a chemical molecule, a peptide, a protein, an aptamer or an antibody.

7. The method according to claim 1, wherein the TRPA1 receptor antagonist is selected from the group consisting of:
(a) the compounds of formula (I), their salts, their tautomers and their enantiomers: 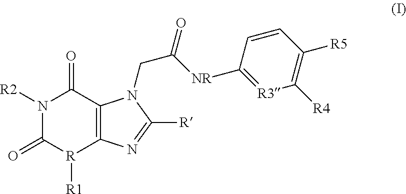
wherein R, R′, R1, R2 and R5 are identical or different, and are selected from a hydrogen atom and a C1 to C12 alkyl radical;
R3 is a carbon atom or a heteroatom; and
R4 is a hydrogen atom or a radical (A): 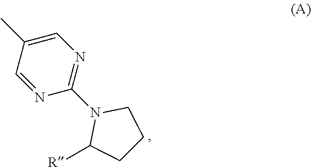
wherein R″ is a hydrogen atom or a C1-C12 alkyl radical;
(b) the compounds of formula (II), their salts, their tautomers and their enantiomers: 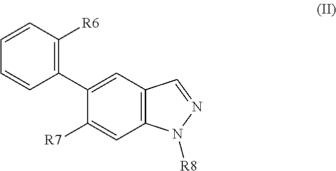
wherein R6, R7 and R8 are identical or different, and are chosen from a hydrogen atom, a C1 to C12 alkyl radical, a —CF3 radical and a —O—R9 radical, wherein R9 is a C1 to C12 alkyl radical;
(c) the compounds of formula (III), their salts, their tautomers and their enantiomers: 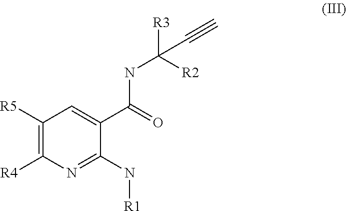
wherein
R1 is (C1-C6) alkyl, cyclo(C3-C6) alkyl, halo(C1-C6) alkyl, or phenyl, wherein the cyclo(C3-C6) alkyl, or phenyl is unsubstituted or substituted with 1 or 2 substituent(s) each independently being halogen, (C1-C2) alkyl or (C1-C2)alkoxy;
R2 is (C1-C2) alkyl;
R3 is (C1-C2) alkyl;
R4 is H, halogen, (C1-C2)alkoxy or halo(C1-C2)alkyl; and
R5 is H, halogen, (C1-C2)alkyl, (C1-C2)alkoxy, halo(C1-C2)alkyl, halo (C1-C2)alkoxy or (C1-C2)alkylthio;
(d) the compounds of formula (IV), their salts, their tautomers and their enantiomers: 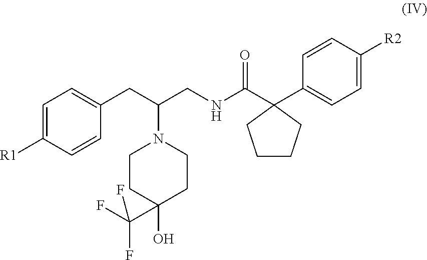
wherein R1 and R2 are each independently selected from H, halogen, OH, CH3, CF3 OCH3 and CN;
(e) the compounds of formula (V), their salts, their tautomers and their enantiomers: 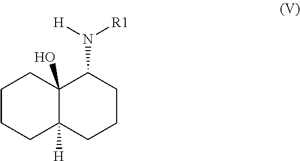
wherein R1 is selected from the group consisting of (CHR2)nC5-C10 heterocyclyl, (CHR2)nC6-C10 aryl, (CHR2)nC3-C10 cycloalkyl, or (C1-C6) alkyl; said alkyl, cycloalkyl, heterocyclyl and aryl being optionally substituted with 1 to 3 groups of R3; and n is an integer from 0 to 20; and
R2 and R3, when present, are each independently methyl or halogen;
(f) the compounds of formula (VI), their salts, their tautomers and their enantiomers: 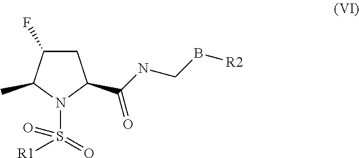
wherein
B is a 6-membered heteroaryl, wherein the 6-membered heteroaryl is unsubstituted or substituted with one or more groups independently selected from halogen, CN, (C1-C6) alkyl, (C1-C6) haloalkyl, O(C1-C6)alkyl, O(C1-C6)haloalkyl, 5 or 6-membered heteroaryl, (C3-C7)cycloalkyl, and 4, 5, 6 or 7-membered heterocyclyl; and wherein any of the 5 or 6-membered heteroaryl, (C3-C7)cycloalkyl, or 4, 5, 6 or 7-membered heterocyclyl groups is unsubstituted or substituted with one or more groups independently selected from halogen, CN, (C1-C6) alkyl, (C1-C6)haloalkyl, O(C1-C6)alkyl and O(C1-C6)haloalkyl;
R1 is phenyl or heteroaryl, wherein each phenyl or heteroaryl is unsubstituted or substituted with one or more groups independently selected from halogen, CN, (C1-C6)alkyl and (C1-C6)haloalkyl;
R2 is phenyl, (C3-C7)cycloalkyl, 5 or 6-membered heteroaryl, or 4, 5, 6 or 7-membered heterocycle, wherein any phenyl, (C3-C7)cycloalkyl, 5 or 6-membered heteroaryl, or 4, 5, 6 or 7-membered heterocycle is optionally substituted with one or more groups independently selected from halogen, CN, SF5, (C1-C6)alkyl, (C1-C6)haloalkyl, O(C1-C6)alkyl or O(C1-C6)haloalkyl;
(g) the compounds of formula (VII), their salts, their tautomers and their enantiomers: 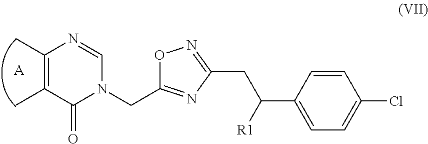
wherein
R1 is selected from H, OH, OMe or halogen; and
A is selected from the following: 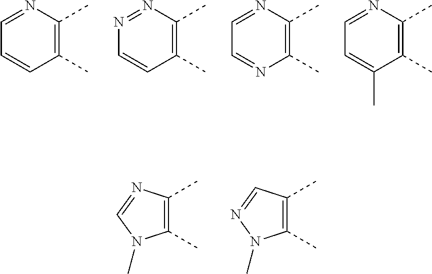
(h) the compounds of formula (VIII), their salts, their tautomers and their enantiomers: 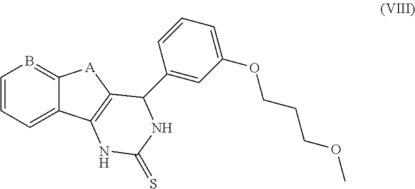
wherein A is:
(i) a heteroatom, or
(ii) —C(═O)—,
and B is:
(i) a carbon atom, optionally linked to a halogen, or
(ii) a heteroatom; and
(i) the compound of the following formula (IX), their salts, their tautomers and their enantiomers:

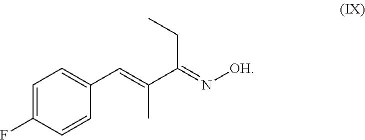

8. The method according to claim 7, wherein the C1 to C12 alkyl radical is a linear or branched, substituted or unsubstituted, optionally cyclic, alkyl radical comprising from 1 to 12 carbon atoms.

9. The method according to claim 8, wherein the alkyl radical comprising from 1 to 12 carbon atoms is selected from the group consisting of methyl, ethyl, n-propyl, i-propyl, cyclopropylmethyl, n-butyl, 2-butyl, t-butyl, n-pentyl, i-pentyl and n-hexyl radicals.

10. The method according to claim 8, characterized in that the heteroatom is an atom selected from oxygen, nitrogen, sulfur or phosphorus.

11. The method according to claim 7, wherein the halogen is a bromide, chloride, fluoride or iodine atom.

12. The method according to claim 7, wherein the C6-C10 aryl is an aromatic carbon cycle, comprising from 6 to 10 carbon atoms.

13. The method according to claim 12, wherein the aryl is a phenyl.

14. The method according to claim 7, wherein the heteroaryl is an aromatic heterocycle.

15. The method according to claim 14, wherein the aromatic heterocycle is pyridine or pyrimidine.

16. The method according to claim 1, wherein the TRPA1 receptor antagonist is of formula (I) as follows:
R and R′ are each hydrogen;
R1 and R2 are each a C1 to C12 alkyl radical;
R3 is a carbon atom or a heteroatom;
R4 is a hydrogen atom or a radical (A): 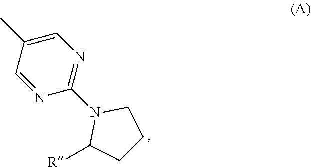
wherein R″ is a C1 to C12 alkyl radical; and
R5 is a hydrogen atom or a C1 to C12 alkyl radical.

17. The method according to claim 16, wherein the C1 to C12 alkyl radical is methyl; the C1 to C12 alkyl radical of R5 is a 2-butyl radical; and the heteroatom is a nitrogen atom.

18. The method according to claim 1, wherein the TRPA1 receptor antagonist is of formula (II) as follows:
R6 is —CF3;
R7 is a C1 to C12 alkyl radical, or R7 is a radical —O—R9, where R9 is a C1 to C12 alkyl radical; and
R8 is hydrogen.

19. The method according to claim 18, wherein the C1 to C12 alkyl radical of R7 is a methyl; and the C1 to C12 alkyl radical of R9 is cyclopropylmethyl.

20. A method for reinforcing the skin barrier function in a patient suffering from atopic dermatitis, comprising administering to the subject a TRPA1 receptor antagonist.
